(19) United States
(12) Patent Application Publication
(10) Pub. No.: US 2018/0050976 A1
(43) Pub. Date: Feb. 22, 2018

(54) Practical Processes for Producing Fluorinated alpha-Ketocarboxylic Esters and Analogues Thereof

(71) Applicant: Central Glass Company, Limited, Ube-shi, Yamaguchi (JP)

(72) Inventors: Shoko ISHII, Kawagoe-shi, Saitama (JP); Takako YAMAZAKI, Kawagoe-shi, Saitama (JP); Takashi KASHIWABA, Kawagoe-shi, Saitama (JP); Ryuichi OKAMOTO, Kawagoe-shi, Saitama (JP); Akihiro ISHII, Kawagoe-shi, Saitama (JP)

(21) Appl. No.: 15/551,160

(22) PCT Filed: Jan. 22, 2016
(86) PCT No.: PCT/JP2016/051771

§ 371 Date: Aug. 15, 2017

(30) Foreign Application Priority Data
Feb. 16, 2015 (JP) 2015-027820

(51) Int. Cl. C07C 67/313; C07C 67/31

(52) U.S. Cl. CPC C07C 67/313; C07C 67/31

(57) ABSTRACT

It is possible to produce a fluorine-containing α-ketocarboxylic ester hydrate by reacting a fluorine-containing α-hydroxycarboxylic ester with sodium hypochlorite or calcium hypochlorite of 21 mass % or greater in mass percentage of composition. Furthermore, it is possible to produce a fluorine-containing α-ketocarboxylic ester by reacting the hydrate with a dehydrating agent. Furthermore, it is possible to produce a fluorine-containing α-ketocarboxylic ester hemiketal by reacting the fluorine-containing α-ketocarboxylic ester hydrate with a lower alcohol or a trialkyl orthocarboxylate. Moreover, it is possible to produce a fluorine-containing α-ketocarboxylic ester by reacting the hemiketal with a dealcoholization agent.

### TECHNICAL FIELD

The present invention relates to a practical process for producing fluorine-containing α-ketocarboxylic esters, which are important as intermediates of medicines and agricultural chemicals.

### BACKGROUND TECHNOLOGY

Fluorine-containing α-ketocarboxylic esters are important compounds as intermediates of medicines and agricultural chemicals. As typical processes for producing fluorine-containing α-ketocarboxylic esters, those of Non-patent Publications 1 to 4 can be cited. Non-patent Publication 1 discloses a process for producing a 3,3,3-trifluoropyruvate from hexafluoropropene-1,2-oxide. Non-patent Publication 2 discloses a process for producing a 3,3-difluoropyruvate, using 3,3,3-trifluorolactate derivative's dehydrofluorination and tautomerism as key reactions. Non-patent Publication 3 discloses a process for producing a 3,3-difluoropyruvate, using a 2-trifluoroacetylfuran derivative's reductive defluorination and an oxidative decomposition of the furan moiety as key reactions. Non-patent Publication 4 discloses a process of oxidizing a fluorine-containing α-hydroxycarboxylate by Dess-Martin reagent to produce the corresponding fluorine-containing α-ketocarboxylate.

On the other hand, Non-patent Publication 5 discloses a process of oxidizing an alcohol having a trifluoromethyl group at α-position by Dess-Martin reagent to produce the corresponding trifluoromethyl ketone. Furthermore, Patent Publication 1 discloses a process for producing a trifluoromethyl ketone by reacting an alcohol having a trifluoromethyl group at α-position with an aqueous solution of a hypohalous acid of a low content (1 to 20 mass %).

### PRIOR ART PUBLICATIONS

### Patent Publication

Patent Publication 1: Japanese Patent Application Publication Heisei 7-506337.

### Non-Patent Publications

Non-patent Publication 1: Journal of Fluorine Chemistry (Netherlands), 2002, volume 115, pages 67-74.
Non-patent Publication 2: Journal of Organic Chemistry (United Stated), 1996, volume 61, pages 7521-7528.
Non-patent Publication 3: Journal of Fluorine Chemistry (Netherlands), 2009, volume 130, pages 682-683.
Non-patent Publication 4: Journal of Organic Chemistry (United Stated), 1995, volume 60, pages 5174-5179.
Non-patent Publication 5: Tetrahedron (United Kingdom), 1991, volume 47, pages 3207-3258.

### DISCLOSURE OF THE INVENTION

### Task to be Solved by the Invention

It is a task of the present invention to provide a practical process for producing fluorine-containing α-ketocarboxylic esters, which are important as intermediates of medicines and agricultural chemicals.

Non-patent Publication 1 discloses a process for producing 3,3,3-trifluoropyruvate, which is high in practicability. It is, however, specialized in the present compound. It was not possible to successfully be applied to analogous compounds such as 3,3-difluoropyruvate. In Non-patent Publication 2, a side reaction was predominant in tautomerism to cause a low yield. In Non-patent Publication 3, an extremely low temperature condition was necessary, causing difficulty in scaling up.

In view of problems of conventional production processes, it is considered that a style of oxidizing a fluorine-containing α-hydroxycarboxylate, which is relatively easy in availability, is superior from the viewpoint of practicability. However, an oxidizing agent achieving good results in such alcohol's oxidation is limited to Dess-Martin reagent, which is expensive and of which danger in handling is pointed out. Therefore, it was not suitable for scaling up (Non-patent Publication 4).

The process described in Patent Publication 1 is a remarkably practical production process, as compared with processes of oxidizing analogous raw materials by Dess-Martin reagent, which are described in Non-patent Publication 5. However, even if a fluorine-containing α-hydroxycarboxylate, which is a raw material of the present invention, is subjected to a typical reaction condition of Patent Publication 1 (see Example 6 of this publication), the amount of the target product contained in a collected organic layer was small, and it was found that Patent Publication 1 cannot provide a practical process for producing the target compound of the present invention (see Comparative Example 1). To the contrary, even though the raw material, which is claimed in Patent Publication 1, and its analogous raw materials are subjected to a preferable reaction condition of the present invention, it was not possible to obtain satisfactory results (see Comparative Examples 2 and 3).

Therefore, it is a specific task of the present invention to find a novel production process for obtaining a fluorine-containing α-ketocarboxylic ester by oxidizing a fluorine-containing α-hydroxycarboxylic ester.

### Means for Solving the Task

As a result of an eager study to solve the task, the present inventors have newly found that a reaction of a fluorine-containing α-hydroxycarboxylic ester represented by the general formula [1] (hereinafter referred to as the compound [1]) with sodium hypochlorite or calcium hypochlorite of 21 mass % or greater in mass percentage of composition is capable of producing a fluorine-containing α-ketocarboxylic ester hydrate represented by the general formula [2] (hereinafter referred to as the compound [2]).

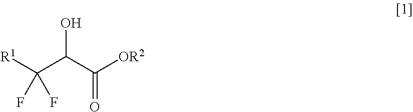

[In the formula, R¹ represents a hydrogen atom, halogen atom or haloalkyl group, and R² represents an alkyl group or substituted alkyl group.]

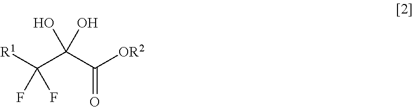

[In the formula, R¹ and R² are identical with those of the general formula [1].]

A hypochlorite disclosed in Patent Publication 1 as an oxidizing agent is used even in the present invention, but the content of hypochlorite is clearly different, and the raw material substrate as the target is clearly different.

The original oxidation product is considered to be a fluorine-containing α-ketocarboxylic ester represented by the general formula [3] (hereinafter referred to as the compound [3]), but it is obtained as the compound [2] resulting from hydration of α-keto group by water derived from the oxidation agent and water produced by the reaction as a by-product in the same amount. Therefore, the present invention contains the step of dehydrating the compound [2] into the compound [3].

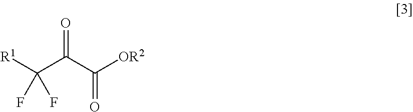

[In the formula, R¹ and R² are identical with those of the general formula [1].]

On the other hand, it is possible to easily convert the compound [2] into a fluorine-containing α-ketocarboxylic ester hemiketal represented by the general formula [5] (hereinafter referred to as the compound [5]) (see Examples 5 and 6). Furthermore, in some cases, a more efficient collection is possible via the compound [5], as compared with a direct conversion of the compound [2] into the compound [3] (see the after-mentioned Examples 11 and 12, too).

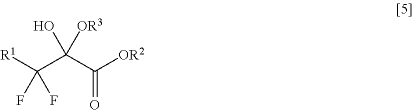

[In the formula, R¹ and R² are identical with those of the general formula [1], and R³ represents a C₁₋₄ alkyl group.]

Furthermore, it was found that the compound [5] has a reactivity equivalent to that of the compound [3] (see Reference Examples 4-6) and is superior to the compound [3] in a long term storage. Thus, the compound [5] is one that is capable of effectively functioning as a synthesis equivalent of the compound [3].

The target of the present invention is a range shown in Scheme 1. Step A is an oxidation step to produce the compound [2] by reacting the compound [1] with sodium hypochlorite or calcium hypochlorite of 21 mass % or greater in mass percentage of composition. Step B is a dehydration step to produce the compound [3] by reacting the compound [2], produced by Step A, with a dehydrating agent. Step C is a hemiketal formation step to produce the compound [5] by reacting the compound [2], produced by Step A, with a lower alcohol or a trialkyl orthocarboxylate. Step D is a dealcoholization step to produce the compound [3] by reacting the compound [5], produced by Step C, with a dealcoholization agent. Incidentally, the compound [3] returns instantly to the compound [2] or the compound [5] by contact with water or a lower alcohol. Furthermore, the compound [5] also easily returns to the compound [2] by contact with water.

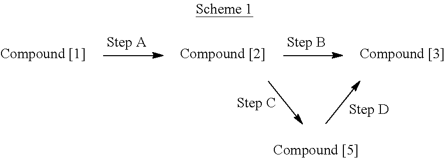

Of sodium hypochlorite or calcium hypochlorite of 21 mass % or greater in mass percentage of composition, one of 31 mass % or greater is preferable, and NaClO.5H₂O or Ca(ClO)₂.nH₂O [n represents an integer of 0 to 3] is particularly preferable, to conduct a desired reaction with good yield.

The present invention can suitably be applied to production of 3,3-difluoropyruvates too, which were limited in the practical production process. Therefore, 3,3-difluorolactates can be cited as preferable modes of the compound [1].

In Step A, it is possible to smoothly conduct a desired reaction by conducting the reaction in the presence of a phase-transfer catalyst. Furthermore, in Step A, it is also possible to conduct the reaction without using a reaction solvent. This contributes to high productivity and waste reduction from the industrial viewpoint.

It was also found that the compound [3] can be produced by reacting the compound [2], produced by Step A, with a dehydrating agent.

As the dehydrating agent, diphosphorus pentoxide and concentrated sulfuric acid are preferable, to collect the compound [3] with good yield.

Furthermore, it was also found that the compound [5] can be produced by reacting the compound [2], produced by Step A, with a lower alcohol (hereinafter referred to as Step C-1).

As the lower alcohol, methanol and ethanol are preferable. With this, it is possible to lower boiling point of the compound [5] to be obtained. Thus, a distillation purification can be conducted even if it has a thermally unstable hemiketal structure.

As a side reaction of Step C-1, a transesterification (ester moiety (—CO₂R²) of the compound [2]+a lower alcohol (R³OH)→—CO₂R³+R²OH) may occur, but it is possible to substantially avoid this by using the same alkyl group for R² of the compound [2] and R³ of the lower alcohol. Thus, it becomes a preferable mode.

In Step C-1, it is possible to conduct a desired reaction with a short period of time by conducting the reaction in the presence of an acid catalyst.

Furthermore, it is possible to conduct a desired reaction with good reproducibility by reacting the compound [2], produced by Step A, with a trialkyl orthocarboxylate (hereinafter referred to as Step C-2). It is possible to change equilibrium between the compound [2] and the compound [5] toward the side of the compound [5] by consuming water existing in the reaction system [for example, R⁴C(OR³)₃+H₂O→R⁴CO₂R³+2R³OH].

As the trialkyl orthocarboxylate, trimethyl orthoformate, triethyl orthoformate, trimethyl orthoacetate and triethyl orthoacetate are preferable. With this, it is possible to lower boiling point of the compound [5] to be obtained. Thus, a distillation purification can be conducted even if it has a thermally unstable hemiketal structure.

As a side reaction of Step C-2, a transesterification (ester moiety (—CO₂R²) of the compound [2]+a lower alcohol (R³OH) that is formed in the reaction system→—CO₂R³+R²OH) may occur, but it is possible to substantially avoid this by using the same alkyl group for R² of the compound [2] and R³ of the trialkyl orthocarboxylate. Thus, it becomes a preferable mode.

In Step C-2, it is possible to conduct a desired reaction with a short period of time by conducting the reaction in the presence of an acid catalyst.

It was also found that the compound [3] can be produced by reacting the compound [5], produced by Step C-2, with a dealcoholization agent.

As the dealcoholization agent, diphosphorus pentoxide and concentrated sulfuric acid are preferable, to collect the compound [3] with good yield.

That is, the present invention provides the following [Invention 1] to [Invention 17].

[Invention 1]

A process for producing a fluorine-containing α-ketocarboxylic ester hydrate represented by the general formula [2] by reacting a fluorine-containing α-hydroxycarboxylic ester represented by the general formula [1] with sodium hypochlorite or calcium hypochlorite of 21 mass % or greater in mass percentage of composition.

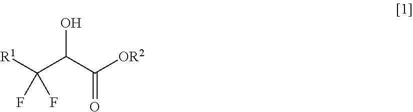

[In the formula, R¹ represents a hydrogen atom, halogen atom or haloalkyl group, and R² represents an alkyl group or substituted alkyl group.]

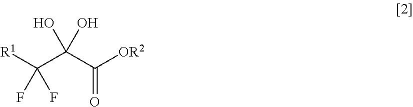

[In the formula, R¹ represents a hydrogen atom, halogen atom or haloalkyl group, and R² represents an alkyl group or substituted alkyl group.]

[Invention 2]

The process according to Invention 1, which is characterized in that the reaction is conducted with sodium hypochlorite or calcium hypochlorite of 31 mass % or greater in mass percentage of composition.

[Invention 3]

The process according to Invention 1, which is characterized in that the reaction is conducted with NaClO.5H₂O or Ca(ClO)₂.nH₂O [in the formula, n represents an integer of 0 to 3].

[Invention 4]

The process according to any of Inventions 1 to 3, which is characterized in that R¹ of the fluorine-containing α-hydroxycarboxylic ester represented by the general formula [1] is a hydrogen atom.

[Invention 5]

The process according to any of Inventions 1 to 4, which is characterized in that the reaction is conducted in the presence of a phase-transfer catalyst.

[Invention 6]

The process according to any of Inventions 1 to 5, which is characterized in that the reaction is conducted without using a reaction solvent.

[Invention 7]

A process for producing a fluorine-containing α-ketocarboxylic ester represented by the general formula [3] by producing a fluorine-containing α-ketocarboxylic ester hydrate represented by the general formula [2] by the process according to any of Inventions 1 to 6 and then reacting the ester hydrate with a dehydrating agent.

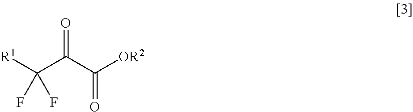

[In the formula, R¹ represents a hydrogen atom, halogen atom or haloalkyl group, and R² represents an alkyl group or substituted alkyl group.]

[Invention 8]

The process according to Invention 7, which is characterized in that the dehydrating agent is diphosphorus pentoxide or concentrated sulfuric acid.

[Invention 9]

A process for producing a fluorine-containing α-ketocarboxylic ester hemiketal represented by the general formula [5] by producing a fluorine-containing α-ketocarboxylic ester hydrate represented by the general formula [2] by the process according to any of Inventions 1 to 6 and then reacting the ester hydrate with a lower alcohol represented by the general formula [4].

R³OH [4]

[In the formula, R³ represents a C₁₋₄ alkyl group.]

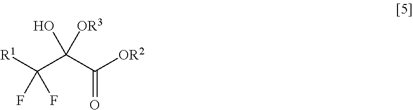

[In the formula, R¹ represents a hydrogen atom, halogen atom or haloalkyl group, R² represents an alkyl group or substituted alkyl group, and R³ represents a C₁₋₄ alkyl group.]

[Invention 10]

The process according to Invention 9, which is characterized in that R³ of the lower alcohol represented by the general formula [4] is a methyl group or ethyl group.

[Invention 11]

The process according to Invention 9 or 10, which is characterized in that R² of the fluorine-containing α-ketocarboxylic ester hydrate represented by the general formula [2] and R³ of the lower alcohol represented by the general formula [4] are identical alkyl groups.

[Invention 12]

A process for producing a fluorine-containing α-ketocarboxylic ester hemiketal represented by the general formula [5] by producing a fluorine-containing α-ketocarboxylic ester hydrate represented by the general formula [2] by the process according to any of Inventions 1 to 6 and then reacting the ester hydrate with a trialkyl orthocarboxylate represented by the general formula [6].

R⁴C(OR³)₃ [6]

[In the formula, R³ represents a C₁₋₄ alkyl group, and R⁴ represents a hydrogen atom, methyl group or ethyl group.]

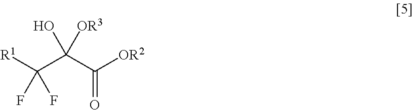

[In the formula, R¹ represents a hydrogen atom, halogen atom or haloalkyl group, R² represents an alkyl group or substituted alkyl group, and R³ represents a C₁₋₄ alkyl group.]

[Invention 13]

The process according to Invention 12, which is characterized in that R³ of the trialkyl orthocarboxylate represented by the general formula [6] is a methyl group or ethyl group.

[Invention 14]

The process according to Invention 12 or 13, which is characterized in that R² of the fluorine-containing α-ketocarboxylic ester hydrate represented by the general formula [2] and R³ of the trialkyl orthocarboxylate represented by the general formula [6] are identical alkyl groups.

[Invention 15]

The process according to any of Inventions 9 to 14, which is characterized in that the reaction is conducted in the presence of an acid catalyst.

[Invention 16]

A process for producing a fluorine-containing α-ketocarboxylic ester represented by the general formula [3] by producing a fluorine-containing α-ketocarboxylic ester hemiketal represented by the general formula [5] by the process according to any of Inventions 9 to 15 and then reacting the ester hemiketal with a dealcoholization agent.

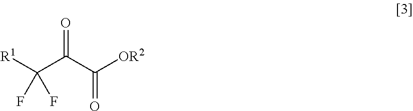

[In the formula, R¹ represents a hydrogen atom, halogen atom or haloalkyl group, and R² represents an alkyl group or substituted alkyl group.]

[Invention 17]

The process according to Invention 16, which is characterized in that the dealcoholization agent is diphosphorus pentoxide or concentrated sulfuric acid.

The present invention provides an advantageous effect that a fluorine-containing α-hydroxycarboxylic ester can efficiently be produced with high yield by suitably combining the raw material and the reaction condition.

### MODE FOR IMPLEMENTING THE INVENTION

Regarding the details of the present invention, the oxidation step, the dehydration step, the hemiketal formation step, and the dealcoholization step are explained in the following in this order.

1. Oxidation Step

The present step is a step for producing the fluorine-containing α-ketocarboxylic ester hydrate represented by the general formula [2] by reacting the fluorine-containing α-hydroxycarboxylic ester represented by the general formula [1] with sodium hypochlorite or calcium hypochlorite of 21 mass % or greater in mass percentage of composition.

R¹ of the fluorine-containing α-hydroxycarboxylic ester represented by the general formula [1] represents a hydrogen atom, a halogen atom, or a haloalkyl group. The halogen atom is a fluorine atom, chlorine atom, bromine atom, or iodine atom. The haloalkyl group has any number and any combination of the halogen atom(s) on any carbon atom(s) of a C₁₋₁₂ straight-chain, branched chain or cyclic (in case that the number of carbon atoms is 3 or greater) alkyl group. In particular, hydrogen atom, fluorine atom, chlorine atom, bromine atom, and a C₁₋₆ haloalkyl group are preferable, and hydrogen atom is particularly preferable.

R² of the fluorine-containing α-hydroxycarboxylic ester represented by the general formula [1] represents an alkyl group or a substituted alkyl group. The alkyl group is C₁₋₈ straight-chain, branched chain or cyclic (in case that the number of carbon atoms is 3 or greater). The substituted alkyl group has any number and any combination of substituent group(s) on any carbon atom(s) of the alkyl group. Such substituent group is the above-mentioned halogen atom or a C₁₋₄ alkoxy group. The alkyl moiety of the alkoxy group is straight-chain, branched chain or cyclic (in case that the number of carbon atoms is 3 or greater). In particular, a C₁₋₄ alkyl group is preferable, and methyl group and ethyl group are particularly preferable.

It is possible to prepare the fluorine-containing α-hydroxycarboxylic ester represented by the general formula [1] with reference to Japanese Patent Application Publications 1993-279314 and 2004-018503, International Publication 2014-078220 (hereinafter, referred to as Patent Publication 2), Non-patent Publication 4, etc. (see Reference Example 1). Even novel compounds in the narrow sense, in which substituent groups of R¹ and R² are slightly different, can similarly be prepared. In particular, a compound having a preferable combination of R¹ and R² is preferable, and a compound having a particularly preferable combination of R¹ and R² are particularly preferable.

Chemical formulas of sodium hypochlorite and calcium hypochlorite are respectively represented by NaCiO and Cl(ClO)₂. In many cases, sodium hypochlorite and calcium hypochlorite are used in the form of hydrate or aqueous solution. Furthermore, in production, it may contain an inorganic salt having no oxidation activity.

Sodium hypochlorite or calcium hypochlorite of 21 mass % or greater in mass percentage of composition means that a component as NaCiO or CA(ClO)₂ is contained by 21 mass % or greater. Specifically, it is possible to cite compounds having mass percentages exemplified as follows. In particular, one of 31 mass percent or greater is preferable, and NaCiO.5H₂O and Ca(ClO)₂.nH₂O are particularly preferable. n of Ca(ClO)₂.nH₂O represents an integer of 0 to 3. As mass percentage is exemplified, NaCiO.5H₂O becomes 45 mass % by NaClO's molecular weight (74.4)÷NaCiO.5H₂O's molecular weight (164.5)×100. By similar calculations, Ca(ClO)₂.H₂O, Ca(ClO)₂.2H₂O, Ca(ClO)₂.3H₂O, and Ca(ClO)₂—CaCl₂.2H₂O [CaCl(ClO).H₂O] respectively become 89 mass %, 80 mass %, 73 mass %, and 49 mass %. Naturally, a sodium hypochlorite aqueous solution of 12 mass % and Ca(ClO)₂ respectively become 12 mass % and 100 mass %. In terms of the content of sodium hypochlorite or calcium hypochlorite, the claims of the present invention cover even a case in which the reaction is conducted by intentionally adding an additive or the like having substantially no impact on the oxidation reaction itself (or having no oxidation activity) and by adjusting the apparent content of the oxidation agent to less than 21 mass %.

In the case of NaCiO.5H₂O preferable as the oxidation agent, it is possible to use one having an industrial product grade, and it is superior to a low-content, sodium hypochlorite aqueous solution in long-term storage stability. Therefore, it is advantageous in an industrial implementation.

It suffices to use sodium hypochlorite or calcium hypochlorite by 0.7 mol or greater, preferably 0.8 to 7 mol, particularly preferably 0.9 to 5 mol, as the NaClO or Ca(ClO)₂ component, relative to 1 mol of the fluorine-containing α-hydroxycarboxylic ester represented by the general formula [1].

In many cases, the present step becomes a heterogeneous reaction. Therefore, according to need, it is also possible to conduct the reaction in the presence of a phase-transfer catalyst. Naturally, it is not always necessary to use a phase-transfer catalyst by adopting suitable reaction conditions.

The phase-transfer catalyst is not particularly limited. It is a quaternary ammonium salt, a phosphonium salt, a polyether (polyethylene glycol or crown ether), or the like. In particular, a quaternary ammonium salt is preferable, and tetra-n-butylammonium bromide and tetra-n-butylammonium hydrogen sulfate are particularly preferable.

The quaternary ammonium salt is represented by the general formula [7].

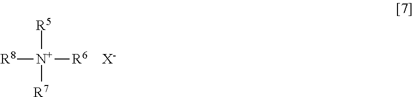

[In the formula, R⁵, R⁶, R⁷ and R⁸ respectively independently represent alkyl groups or aralkyl groups, and X⁻ represents a halide ion or hydrogensulfate ion (HSO₄⁻).]

The alkyl group is a C₁₋₁₂ straight-chain, branched chain or cyclic (in case that the number of carbon atoms is 3 or greater). The aralkyl group is C₁₋₁₂, and the alkyl moiety is straight-chain, branched chain, or cyclic (in case that the number of carbon atoms is 9 or greater). The halide ion is a fluoride ion, chloride ion, bromide ion, or iodide ion.

It suffices to use the phase-transfer catalyst by 0.7 mol or less, preferably 0.0001 to 0.5 mol, particularly preferably 0.0005 to 0.3 mol, relative to 1 mol of the fluorine-containing α-hydroxycarboxylic ester represented by the general formula [1].

The reaction solvent is not particularly limited. It is an aliphatic hydrocarbon series such as n-hexane, cyclohexane or n-heptane, an aromatic hydrocarbon series such as toluene, xylene or mesitylene, a halogen series such as methylene chloride, chloroform or 1,2-dichloroethane, an ether series such as tetrahydrofuran, tert-butyl methyl ether or 1,2-dimethoxyethane, an ester series such as ethyl acetate, n-butyl acetate or propylene glycol monomethyl ether acetate, an amide series such as N,N-dimethylformamide, N,N-dimethylacetamide or 1,3-dimethyl-2-imidazolidinone, or a nitrile series such as acetonitrile, propionitrile or benzonitrile. In particular, aromatic hydrocarbon series, halogen series, ether series, ester series and nitrile series are preferable, and aromatic hydrocarbon series, ester series and nitrile series are particularly preferable. These reaction solvents can be used singly or in combination. It is also possible to conduct the present step without using reaction solvent. In some cases, neat reaction becomes a preferable mode.

Usage of the reaction solvent may be 0.01 L (liter) or greater, preferably 0.02-7 L, particularly preferably 0.03-5 L, relative to 1 mol of the fluorine-containing α-hydroxycarboxylic ester represented by the general formula [1].

The reaction temperature may be +150° C. or lower, preferably +125 to −50° C., particularly preferably +100 to −25° C.

The reaction time may be 48 hours or shorter. It depends on the raw material substrate and the reaction condition. Therefore, it is preferable to follow the reaction progress condition by an analytical means such as gas chromatography, liquid chromatography, nuclear magnetic resonance, etc. It is preferable to determine the time when the decrease of the raw material substrate is almost not found, as terminal point.

It is possible to obtain the fluorine-containing α-ketocarboxylic ester hydrate represented by the general formula [2] by adopting a normal operation in organic synthesis in the post-treatment. In case that the reaction is conducted without using reaction solvent and that boiling point of the target product is sufficiently low, a direct distillation recovery operation from the reaction-terminated liquid is simple and easy (see Example 4). According to need, the recovered crude product can be purified to high purity by fractional distillation, recrystallization, column chromatography, etc.

The present step and the hemiketal formation reaction can also be conducted as one-pot reactions, and it is a preferable mode of the present invention (see Example 8).

R¹ and R² of the fluorine-containing α-ketocarboxylic ester hydrate represented by the general formula [2] derive from R¹ and R² of the fluorine-containing α-hydroxycarboxylic ester represented by the general formula [1].

The compound [1] as the raw material of the present invention has an ester group that is easily hydrolysable. Therefore, if it is hydrolyzed prior to the desired oxidation, a fluorine-containing carboxylic acid (R¹CF₂CO₂H) is produced by a considerable amount as a by-product by degradation as a side reaction (see Comparative Example 4). On the other hand, if the target product is hydrolyzed after the oxidation, it turns into a fluorine-containing, α-ketocarboxylic acid hydrate [R¹CF₂C(OH)₂CO₂H], which is highly water soluble, thereby transferring into an aqueous layer. Thus, its recovery in an organic layer becomes difficult. In the present invention, a hypochlorite of high content is used. Therefore, it is possible to not only improve reactivity of the oxidation, but also minimize the amount of water that is introduced into the reaction system to prevent an undesirable hydrolysis of the ester group. Furthermore, as compared with NaClO.5H₂O and Ca(ClO)₂.nH₂O as preferable oxidation agents of the present invention, a 12 mass % sodium hypochlorite aqueous solution, which is used many times in Patent Publication 1, contains large amounts of unnecessary alkali components to have a strong tendency to promote hydrolysis of the ester group (see Comparative Example 1). Thus, it is possible to obtain the desired compound [2] with good yield by using a preferable oxidation agent of the present invention. In the present invention, it is possible to obtain the desired compound [2] with high selectivity. For example, even a target product (R¹ of the compound [2] is a hydrogen atom) having a hydrogen atom at α-position of the carbonyl group (or gem-diol group) is not subjected to chlorination as a side reaction. Therefore, it can preferably be applied to the production of a high purity product of 3,3-difluoropyruvates (see Examples 3 and 4).

Furthermore, in the present invention, it is not always necessary to use a phase-transfer catalyst, which is mentioned as being essential in Patent Publication 1. This contributes to cost reduction and waste reduction from the industrial viewpoint (see Example 1).

Furthermore, in the present invention, it is not always necessary to use a reaction solvent, which is mentioned as being essential in Patent Publication 1. This is advantageous from the industrial viewpoint (see Example 4).

At last, preferable oxidation agents to be used in the present invention are available at low prices in industrial scale and are also safe in handling in industrial scale. Considering that oxidation of analogous raw materials was limited so far to an oxidation agent such as Dess-Martin reagent, high practicability of the present invention can easily be understood.

2. Dehydration Step

The present step is a step for producing the fluorine-containing α-ketocarboxylic ester represented by the general formula [3] by reacting the fluorine-containing α-ketocarboxylic ester hydrate represented by the general formula [2], which was produced by the oxidation step, with a dehydrating agent.

The dehydrating agent is selected from inorganic series such as diphosphorus pentoxide, concentrated sulfuric acid, sodium sulfate, magnesium sulfate, calcium sulfate, calcium chloride, molecular sieve (synthetic zeolite) and silica gel, and organic series such as acetic anhydride, propionic anhydride, benzoic anhydride, succinic anhydride, maleic anhydride, phthalic anhydride, trifluoroacetic anhydride and trifluoromethanesulfonic anhydride. In particular, diphosphorus pentoxide, concentrated sulfuric acid, calcium chloride, acetic anhydride, benzoic anhydride, succinic anhydride, phthalic anhydride, and trifluoroacetic anhydride are preferable, and diphosphorus pentoxide and concentrated sulfuric acid are particularly preferable.

Usage of the dehydrating agent except molecular sieve and silica gel may be 0.1 mol or greater, preferably 0.2-50 mol, particularly preferably 0.3-30 mol, relative to 1 mol of the fluorine-containing α-ketocarboxylic ester hydrate represented by the general formula [2].

Usage of molecular sieve and silica gel may be 0.01 g or greater, preferably 0.02-10 g, particularly preferably 0.03-7 g, relative to 1 g of the fluorine-containing α-ketocarboxylic ester hydrate represented by the general formula [2].

In the case of using an organic-series dehydrating agent, if the reaction is conducted in the presence of an organic base such as tertiary amine or pyridine, it is possible to smoothly conduct the desired reaction. Naturally, it is not always necessary to use an organic base by adopting a suitable reaction condition.

As the organic base, preferable ones are triethylamine, diisopropylethylamine, tri-n-propylamine, tri-n-butylamine, pyridine, lutidines (including all of the positional isomers) and collidines (including all of the positional isomers), and particularly preferable ones are triethylamine, tri-n-butylamine, pyridine and lutidine.

The tertiary amine is represented by the general formula [8].

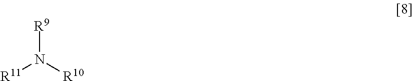

[In the formula, each of R⁹, R¹⁰ and R¹¹ is independently an alkyl group or aralkyl group.]

The alkyl group is a C₁₋₁₂ straight-chain, branched chain or cyclic (in case that the number of carbon atoms is 3 or greater). The aralkyl group is C₁₋₁₂, and the alkyl moiety is straight-chain, branched chain, or cyclic (in case that the number of carbon atoms is 9 or greater).

Usage of the organic base may be 0.1 mol or greater, preferably 0.2-50 mol, particularly preferably 0.3-30 mol, relative to 1 mol of the fluorine-containing α-ketocarboxylic ester hydrate represented by the general formula [2].

The reaction solvent is not particularly limited. It is an aliphatic hydrocarbon series such as n-hexane, cyclohexane or n-heptane, an aromatic hydrocarbon series such as toluene, xylene or mesitylene, a halogen series such as methylene chloride, chloroform or 1,2-dichloroethane, an ether series such as tetrahydrofuran, cyclopentyl methyl ether or diethylene glycol dimethyl ether, an ester series such as ethyl acetate, n-butyl acetate or propylene glycol monomethyl ether acetate, an amide series such as N,N-dimethylformamide, N,N-dimethylacetamide or 1,3-dimethyl-2-imidazolidinone, a nitrile series such as acetonitrile, propionitrile or benzonitrile, or a sulfur series such as dimethylsulfoxide, methyl phenyl sulfoxide or sulfolane. In particular, aromatic hydrocarbon series, halogen series, ether series, ester series and nitrile series are preferable, and aromatic hydrocarbon series, halogen series and ether series are particularly preferable. These reaction solvents can be used singly or in combination. It is also possible to conduct the present step without using reaction solvent. In some cases, neat reaction becomes a preferable mode.

Usage of the reaction solvent may be 0.01 L or greater, preferably 0.02-5 L, particularly preferably 0.03-3 L, relative to 1 mol of the fluorine-containing α-ketocarboxylic ester hydrate represented by the general formula [2].

The reaction temperature may be +200° C. or lower, preferably +175 to −50° C., particularly preferably +150 to −25° C.

The reaction time may be 24 hours or shorter. It depends on the raw material substrate and the reaction condition. Therefore, it is preferable to follow the reaction progress condition by an analytical means such as gas chromatography, liquid chromatography, nuclear magnetic resonance, etc. It is preferable to determine the time when the decrease of the raw material substrate is almost not found, as terminal point.

It is possible to obtain the fluorine-containing α-ketocarboxylic ester represented by the general formula [3] by adopting a normal operation in organic synthesis in the post-treatment. In case that the reaction is conducted without using reaction solvent and that boiling point of the target product is sufficiently low, a direct distillation recovery operation from the reaction-terminated liquid is simple and easy. If the target product is thermally unstable, it is possible to apply an operation in which the produced target product is successively taken out of the reaction system under reduced pressure while adding the raw material to the heated dehydrating agent in a dropwise manner (see Example 11). According to need, the recovered crude product can be purified to high purity by fractional distillation, recrystallization, column chromatography, etc.

R¹ and R² of the fluorine-containing α-ketocarboxylic ester represented by the general formula [3] derive from R¹ and R² of the fluorine-containing α-ketocarboxylic ester hydrate represented by the general formula [2].

3. Hemiketal Formation

The present step is a step for producing the fluorine-containing α-ketocarboxylic ester hemiketal represented by the general formula [5] by reacting the fluorine-containing α-ketocarboxylic ester hydrate represented by the general formula [2] with the lower alcohol represented by the general formula [4] or the trialkyl orthocarboxylate represented by the general formula [6]. In particular, the case of reacting with the lower alcohol represented by the general formula [4] is referred to as the hemiketal formation step-1, and the case of reacting with the trialkyl orthocarboxylate represented by the general formula [6] is referred to as the hemiketal formation step-2.

(Related to the Hemiketal Formation Step-1)

R³ of the lower alcohol represented by the general formula [4] represents a C₁₋₄ alkyl group. The alkyl group is straight-chain, branched chain or cyclic (in case that the number of carbon atoms is 3 or greater). In particular, a C₁₋₃ alkyl group is preferable, and methyl group and ethyl group are particularly preferable.

Usage of the lower alcohol represented by the general formula [4] may be 0.7 mol or greater, preferably 0.8-200 mol, particularly preferably 0.9-150 mol, relative to 1 mol of the fluorine-containing α-ketocarboxylic ester hydrate represented by the general formula [2].

(Related to the Hemiketal Formation Step-2)

R³ of the trialkyl orthocarboxylate represented by the general formula [6] represents a C₁₋₄ alkyl group. The alkyl group is straight-chain, branched chain or cyclic (in case that the number of carbon atoms is 3 or greater). In particular, a C₁₋₃ alkyl group is preferable, and methyl group and ethyl group are particularly preferable.

R⁴ of the trialkyl orthocarboxylate represented by the general formula [6] represents a hydrogen atom, methyl group or ethyl group. In particular, a hydrogen atom and a methyl group are preferable, and a hydrogen atom is particularly preferable.

As the trialkyl orthocarboxylate represented by the general formula [6], a compound having a preferable combination of R³ and R⁴ is preferable, a compound having a particularly preferable combination of R³ and R⁴ is particularly preferable, and trimethyl orthoformate is extremely preferable.

Usage of the trialkyl orthocarboxylate represented by the general formula [6] may be 0.3 mol or greater, preferably 0.4-100 mol, particularly preferably 0.5-75 mol, relative to 1 mol of the fluorine-containing α-ketocarboxylic ester hydrate represented by the general formula [2]. In case that the fluorine-containing α-ketocarboxylic ester hydrate represented by the general formula [2] used as the raw material contains water, it may be used by a somewhat larger quantity in consideration of the water content. In the hemiketal formation step-2, it is also possible to conduct the reaction in the presence of the lower alcohol represented by the general formula [4].

(Common to the Hemiketal Formation Step-1 and the Hemiketal Formation Step-2)

The present step is a step for converting the gem-diol group of the fluorine-containing α-ketocarboxylic ester hydrate represented by the general formula [2] into a hemiketal group, but the above-mentioned transesterification could occur as a side reaction. Naturally, the side reaction can be controlled to minimum by adopting suitable reaction conditions. It can substantially be avoided by using the same alkyl group for R² of the fluorine-containing α-ketocarboxylic ester hydrate represented by the general formula [2] and for R³ of the lower alcohol represented by the general formula [4] or the trialkyl orthocarboxylate represented by the general formula [6]. Therefore, this is a preferable mode (for example, using the same methyl group or ethyl group for R² and R³, and see Examples 5 and 6).

Furthermore, as the fluorine-containing α-ketocarboxylic ester hydrate represented by the general formula [2], which has been produced by the oxidation step, is stored for a long term under a high water content condition, it is decomposed into a fluorine-containing α-ketocarboxylic acid hydrate or hemiketal represented by the general formula [9a] or [9b].

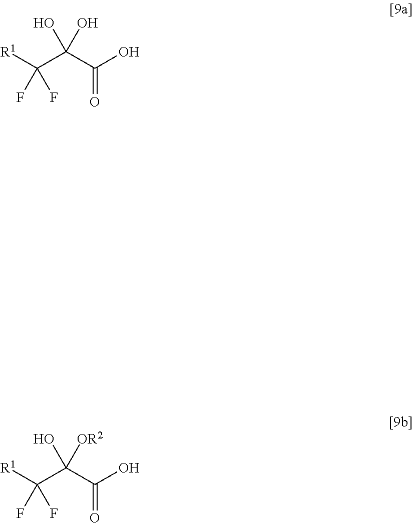

[In the formulas, R¹ and R² are derived from R¹ and R² of the general formula [2].]

The present decomposition product can be converted through the present step into a fluorine-containing α-ketocarboxylate hemiketal represented by the general formula [10a], [10b], [10c] or [5].

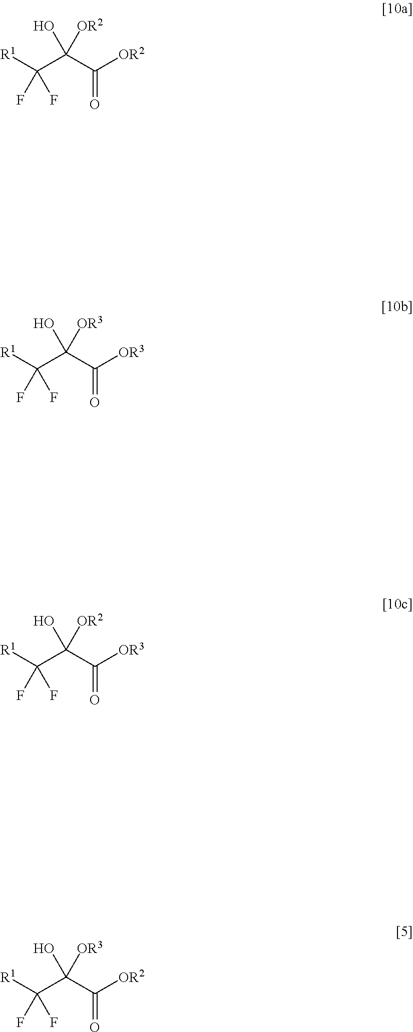

[In the formulas, R¹ and R² are derived from R¹ and R² of the general formula [2], and R³ is derived from R³ of the lower alcohol represented by the general formula [4] or the trialkyl orthocarboxylate represented by the general formula [6].]

The fluorine-containing α-ketocarboxylic acid hydrate or hemiketal represented by the general formula [9a] or [9b] could not be the raw material substrate of the dehydration step or the dealcoholization step, but the fluorine-containing α-ketocarboxylic acid hemiketal represented by the general formula [10a], [10b], [10c] or [5] could be the raw material substrate of the dealcoholization step. In such a case, it is possible to make the fluorine-containing α-ketocarboxylic acid hemiketals represented by the general formula [10a], [10b], [10c] and [5] into the same compound by using the same alkyl group for R² of the fluorine-containing α-ketocarboxylic ester hydrate represented by the general formula [2] and for R³ of the lower alcohol represented by the general formula [4] or the trialkyl orthocarboxylate represented by the general formula [6]. Therefore, it becomes possible to improve recovery of the fluorine-containing α-ketocarboxylate represented by the general formula [3] by going through the compound (the above), and this becomes a preferable mode (for example, using the same methyl group for R² and R³, and see Example 7).

The acid catalyst is not particularly limited. It is selected from inorganic acids such as boric acid, phosphoric acid, hydrogen chloride, hydrogen boride, nitric acid and sulfuric acid, and organic acids such as formic acid, acetic acid, oxalic acid, benzoic acid, benzenesulfonic acid and paratoluenesulfonic acid. In particular, phosphoric acid, hydrogen chloride, sulfuric acid, benzenesulfonic acid and paratoluenesulfonic acid are preferable, and hydrogen chloride, sulfuric acid and paratoluenesulfonic acid are particularly preferable. Naturally, it is not always necessary to use the acid catalyst by adopting suitable reaction conditions (see Example 5).

Usage of the acid catalyst may be 0.7 mol or less, preferably 0.0001 to 0.5 mol, particularly preferably 0.0005 to 0.3 mol, relative to 1 mol of the fluorine-containing α-ketocarboxylic ester hydrate represented by the general formula [2].

The reaction solvent is not particularly limited. It is an aliphatic hydrocarbon series such as n-hexane, cyclohexane or n-heptane, an aromatic hydrocarbon series such as toluene, xylene or mesitylene, a halogen series such as methylene chloride, chloroform or 1,2-dichloroethane, an ether series such as tetrahydrofuran, tert-butyl methyl ether or 1,2-dimethoxyethane, an ester series such as ethyl acetate, n-butyl acetate or propylene glycol monomethyl ether acetate, an amide series such as N,N-dimethylformamide, N,N-dimethylacetamide or 1,3-dimethyl-2-imidazolidinone, or a nitrile series such as acetonitrile, propionitrile or benzonitrile. In particular, aromatic hydrocarbon series, halogen series, ether series, ester series and nitrile series are preferable, and aromatic hydrocarbon series, halogen series and nitrile series are particularly preferable. These reaction solvents can be used singly or in combination. It is also possible to conduct the present step without using reaction solvent. In some cases, neat reaction becomes a preferable mode.

Usage of the reaction solvent may be 0.01 L or greater, preferably 0.02-5 L, particularly preferably 0.03-3 L, relative to 1 mol of the fluorine-containing α-ketocarboxylic ester hydrate represented by the general formula [2].

The reaction temperature may be +150° C. or lower, preferably +125 to −50° C., particularly preferably +100 to −25° C.

The reaction time may be 72 hours or shorter. It depends on the raw material substrate and the reaction condition. Therefore, it is preferable to follow the reaction progress condition by an analytical means such as gas chromatography, liquid chromatography, nuclear magnetic resonance, etc. It is preferable to determine the time when the decrease of the raw material substrate is almost not found, as terminal point.

It is possible to obtain the fluorine-containing α-ketocarboxylic ester hemiketal represented by the general formula [5] by adopting a normal operation in organic synthesis in the post-treatment. In case that the reaction is conducted without using reaction solvent and that boiling point of the target product is sufficiently low, a direct distillation recovery operation from the reaction-terminated liquid is simple and easy (see Examples 5 and 6). According to need, the recovered crude product can be purified to high purity by fractional distillation, recrystallization, column chromatography, etc.

R¹ and R² of the fluorine-containing α-ketocarboxylic ester hemiketal represented by the general formula [5] are derived from R¹ and R² of the fluorine-containing α-ketocarboxylic ester hydrate represented by the general formula [2].

R³ of the fluorine-containing α-ketocarboxylic ester hemiketal represented by the general formula [5] is derived from R³ of the lower alcohol represented by the general formula [4] or the trialkyl orthocarboxylate represented by the general formula [6].

In the above-mentioned distillation recovery or fractional distillation, dealcoholization of the fluorine-containing α-ketocarboxylic ester hemiketal represented by the general formula [5] may occur partially, thereby resulting in recovery as its mixture with the fluorine-containing α-ketocarboxylic ester represented by the general formula [3]. Such case is also included in the claims of the present invention.

4. Dealcoholization Step

The present step is a step for producing the fluorine-containing α-ketocarboxylic ester represented by the general formula [3] by reacting the fluorine-containing α-ketocarboxylic ester hemiketal represented by the general formula [5], which has been produced by the hemiketal formation step, with the dealcoholization agent.

The present step can similarly be conducted with respect to all of the items described in “2. DEHYDRATION STEP” (see Example 11). However, “the dehydrating agent”, “the fluorine-containing α-ketocarboxylic ester hydrate represented by the general formula [2]”, and “produced by the oxidation step” are respectively replaced with “the dealcoholization agent”, “the fluorine-containing α-ketocarboxylic ester hemiketal represented by the general formula [5]”, and “produced by the hemiketal formation step”. Furthermore, preferable modes are identical with respect to all of the items.

### EXAMPLES

In the following, embodiments of the present invention are specifically explained, but the present invention is not limited to these examples.

### [Example 1] Production of ethyl 3,3,3-trifluoropyruvate hydrate (Oxidation of ethyl 3,3,3-trifluorolactate by NaClO.5H₂O)

To 58 mL (1.0 mL/mmol) of acetonitrile, 10 g (58 mmol, 1.0 eq) of ethyl 3,3,3-trifluorolactate was added and dissolved therein. Furthermore, 11 g (67 mmol, 1.2 eq) of NaClO.5H₂O was added, followed by stirring at 20° C. for 30 minutes. As the reaction-terminated liquid was analyzed by ¹⁹F-NMR, conversion was 100%, and selectivity was 98%. To the reaction-terminated liquid, 0.38 g (1.5 mmol, 0.026 eq) of sodium thiosulfate pentahydrate was added, followed by stirring, thereby quenching the remaining oxidation agent. Furthermore, 0.33 g (3.9 mmol, 0.067 eq) of sodium hydrogencarbonate and 10 g (70 mmol, 1.2 eq) of sodium sulfate were added, followed by stirring and then removing solid matter by filtration. As the filtrate was quantified with internal standard method (internal standard substance: α,α,α-trifluorotoluene) by ¹⁹F-NMR, ethyl 3,3,3-trifluoropyruvate hydrate was contained by 56 mmol (quantitative yield: 97%). By a simple distillation (up to 48° C./0.5 kPa) of the filtrate, 7.6 g of ethyl 3,3,3-trifluoropyruvate hydrate was obtained. ¹⁹F-NMR purity was 99% (40 mmol), and the total yield was 69%.

¹H-NMR and ¹⁹F-NMR of ethyl 3,3,3-trifluoropyruvate hydrate are shown in the following.

¹H-NMR (standard substance: tetramethylsilane; solvent: deuterated chloroform), δ ppm; 1.38 (t, 3H), 4.41 (q, 2H), and assignment of proton of gem-diol group was not possible.

¹⁹F-NMR (standard substance: hexafluorobenzene; solvent: deuterated chloroform), δ ppm; 78.6 (s, 3F).

In the present example, a by-product resulting from hydrolysis of the ester group of the target product was not observed at all, and the amount of trifluoroacetic acid as a by-product was less than 1%.

### [Example 2] Production of ethyl 3,3,3-trifluoropyruvate hydrate (Oxidation of ethyl 3,3,3-trifluorolactate by Ca(ClO)₂.3H₂O)

To 6.0 mL (1.0 mL/mmol) of ethyl acetate, 1.0 g (5.8 mmol, 1.0 eq) of ethyl 3,3,3-trifluorolactate and 0.098 g (0.29 mmol, 0.050 eq) of tetra-n-butylammonium hydrogensulfate were added and dissolved therein. Furthermore, 1.2 g (6.1 mmol, 1.1 eq) of Ca(ClO)₂.3H₂O was added, followed by stirring all night at room temperature. As the reaction-terminated liquid was analyzed by ¹⁹F-NMR, conversion was 98%, and selectivity was 93%.

¹H-NMR and ¹⁹F-NMR of ethyl 3,3,3-trifluoropyruvate hydrate were identical with those in Example 1.

In the present example, a by-product resulting from hydrolysis of the ester group of the target product was not observed at all, and the amount of trifluoroacetic acid as a by-product was 3%.

### [Example 3] Production of methyl 3,3-difluoropyruvate hydrate (Oxidation of methyl 3,3-difluorolactate by NaClO.5H₂O)

To 270 mL (1.0 mL/mmol) of ethyl acetate, 38 g (270 mmol, 1.0 eq) of methyl 3,3-difluorolactate and 4.6 g (14 mmol, 0.052 eq) of tetra-n-butylammonium hydrogensulfate were added and dissolved therein. Furthermore, 49 g (300 mmol, 1.1 eq) of NaClO.5H₂O was added, followed by stirring at 15° C. for three hours. As the reaction-terminated liquid was analyzed by ¹⁹F-NMR, conversion was 100%, and selectivity was 95%. To the reaction-terminated liquid, 69 g (55 mmol, 0.20 eq) of 10% sodium sulfite aqueous solution was added, followed by stirring, thereby quenching the remaining oxidation agent. As the recovered organic layer was quantified with internal standard method (internal standard substance: α,α,α-trifluorotoluene) by ¹⁹F-NMR, methyl 3,3-difluoropyruvate hydrate was contained by 200 mmol (quantitative yield: 74%).

¹H-NMR and ¹⁹F-NMR of methyl 3,3-difluoropyruvate hydrate were identical with those in Non-patent Publication 3.

In the present example, a by-product resulting from hydrolysis of the ester group of the target product was not observed at all, and the amount of trifluoroacetic acid as a by-product was 2%. Furthermore, a by-product resulting from chlorination of hydrogen atom at α-position was not observed at all.

### [Example 4] Production of ethyl 3,3-difluoropyruvate hydrate (Oxidation of ethyl 3,3-difluorolactate by NaClO.5H₂O)

To 1.0 g (6.5 mmol, 1.0 eq) of ethyl 3,3-difluorolactate, 0.11 g (0.32 mmol, 0.049 eq) of tetra-n-butylammonium hydrogensulfate and 1.2 g (7.3 mmol, 1.1 eq) of NaClO.5H₂O were added, followed by stirring at 30° C. for 30 minutes. As the reaction-terminated liquid was analyzed by ¹⁹F-NMR, conversion was 96%, and selectivity was 97%. 0.93 g of ethyl 3,3-difluoropyruvate was obtained by Kugelrohr distillation (up to 130° C./0.8 kPa) of the reaction-terminated liquid. ¹⁹F-NMR purity was 94% (5.1 mmol), and the total yield was 78%.

¹H-NMR and ¹⁹F-NMR of ethyl 3,3-difluoropyruvate hydrate are shown in the following.

¹H-NMR (standard substance: tetramethylsilane; solvent: deuterated chloroform), δ ppm; 1.34 (t, 3H), 4.18 (br, 2H), 4.35 (q, 2H), 5.88 (t, 1H).

¹⁹F-NMR (standard substance: hexafluorobenzene; solvent: deuterated chloroform), δ ppm; 26.5 (d, 2F).

In the present example, a by-product resulting from hydrolysis of the ester group of the target product was not observed at all, and the amount of difluoroacetic acid as a by-product was 1%. Furthermore, a by-product resulting from chlorination of hydrogen atom at α-position was not observed at all.

### [Example 5] Production of ethyl 3,3,3-trifluoropyruvate ethylhemiketal (Hemiketal formation from ethyl 3,3,3-trifluoropyruvate hydrate by Ethanol)

To 25 g (540 mmol, 20 eq) of ethanol, 5.0 g (27 mmol, 1.0 eq) of ethyl 3,3,3-trifluoropyruvate hydrate was added, followed by stirring at room temperature for two days. By a simple distillation (up to 44° C./1.5 kPa) of the reaction-terminated liquid, 3.9 g of ethyl 3,3,3-trifluoropyruvate ethylhemiketal was obtained. The molar ratio of the target product to ethanol by ¹H-NMR was 10:1. ¹⁹F-NMR purity was 98% (19 mmol), and the total yield was 70%.

¹⁹F-NMR of ethyl 3,3,3-trifluoropyruvate ethylhemiketal is shown in the following.

¹⁹F-NMR (standard substance: trichlorofluoroethane; solvent: deuterated chloroform), δ ppm; −81.9 (s, 3F).

### [Example 6] Production of methyl 3,3-difluoropyruvate methylhemiketal (Hemiketal Formation from methyl 3,3-difluoropyruvate hydrate by trimethyl orthoformate)

To 2.7 g (25 mmol, 0.96 eq) of trimethyl orthoformate, 4.0 g (26 mmol, 1.0 eq) of methyl 3,3-difluoropyruvate hydrate and 0.25 g (2.5 mmol, 0.096 eq) of sulfuric acid were added, followed by stirring at room temperature for two hours. As the reaction-terminated liquid was analyzed by ¹⁹F-NMR, conversion was 100%. By a simple distillation (up to 59° C./2.1 kPa) of the reaction-terminated liquid, 2.8 g of methyl 3,3-difluoropyruvate methylhemiketal was obtained. The molar ratio of the target product to methanol by ¹H-NMR was 55:8. ¹⁹F-NMR purity was 97% (18 mmol), and the total yield was 69%.

¹⁹F-NMR of methyl 3,3-difluoropyruvate methylhemiketal was identical with that in Non-patent Publication 3.

### [Example 7] Production of methyl 3,3-difluoropyruvate methylhemiketal (Hemiketal Formation from methyl 3,3-difluoropyruvate hydrate, which has been Stored for a Long Term, by Trimethylorthoformate; see Scheme 2)

To a long-term stored product (32 mmol in total, 1.0 eq, containing difluoroacetic acid as a by-product) of methyl 3,3-difluoropyruvate hydrate containing 59 mmol (measured by Karl Fisher method) of water, 9.9 g (93 mmol, 2.9 eq) of trimethyl orthoformate and 0.74 g (7.5 mmol, 0.23 eq) of sulfuric acid were added, followed by stirring all night at room temperature. As the reaction-terminated liquid was quantified with internal standard method (internal standard substance: α,α,α-trifluorotoluene) by ¹⁹F-NMR, methyl 3,3-difluoropyruvate methylhemiketal and methyl difluoroacetate were respectively contained by 26 mmol and 1.1 mmol (27 mmol in total). Water was contained by 0.39 mmol in the reaction-terminated liquid. By a simple distillation (up to 60° C./0.6 kPa) of the reaction-terminated liquid, methyl 3,3-difluoropyruvate methylhemiketal and methyl 3,3-difluoropyruvate were obtained by 19 mmol and 4.7 mmol, respectively. Water was contained by 0.12 mmol in the distillate. It was possible to remove methyl difluoroacetic acid by the simple distillation.

¹⁹F-NMR of methyl 3,3-difluoropyruvate methylhemiketal was equivalent to that of Example 6. ¹H-NMR and ¹⁹F-NMR of methyl 3,3-difluoropyruvate methyl were identical with those in Non-patent Publication 3.

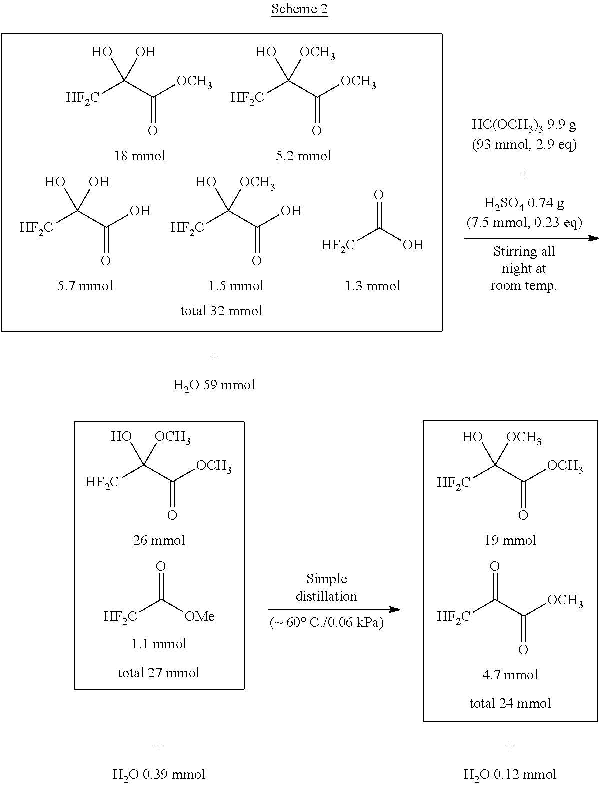

### [Example 8] Production of methyl 3,3-difluoropyruvate methylhemiketal (Oxidation of methyl 3,3-difluorolactate by NaClO.5H₂O→Hemiketal formation from methyl 3,3-difluoropyruvate hydrate by trimethyl orthoformate; One-Pot Reactions)

To 730 mL (350 mL/mol) of acetonitrile, 300 g (2.1 mol, 1.0 eq) of methyl 3,3-difluorolactate and 37 g (0.11 mol, 0.052 eq) of tetra-n-butylammonium hydrogensulfate were added and dissolved. Furthermore, 390 g (2.4 mol, 1.1 eq) of NaClO.5H₂O was added under cooling with ice, followed by stirring at room temperature for 30 minutes. As the reaction-terminated liquid was analyzed by ¹⁹F-NMR, conversion was 100%, and selectivity was 96%. The reaction-terminated liquid was concentrated under reduced pressure to distill acetonitrile out. To the concentration residue, 1600 g (15 mol, 7.1 eq) of trimethyl orthoformate and 11 g (0.11 mol, 0.052 eq) of sulfuric acid were added under cooling with ice, followed by stirring at room temperature for four hours and thirty minutes. As the reaction-terminated liquid was analyzed by ¹⁹F-NMR, conversion was 100%. By a simple distillation (up to 60° C./0.6 kPa) of the reaction-terminated liquid, 270 g of a mixture of methyl 3,3-difluoropyruvate methylhemiketal and methyl 3,3-difluoropyruvate was obtained. The molar ratio of the target product to the dealcoholization product by ¹H-NMR was 86:14. ¹⁹F-NMR purity was 98% or higher (set at 1.6 mol), and the total yield was 76%.

¹⁹F-NMR of methyl 3,3-difluoropyruvate methylhemiketal was equivalent to that of Example 6. ¹H-NMR and ¹⁹F-NMR of methyl 3,3-difluoropyruvate were identical with those in Example 7.

### [Example 9] Production of methyl 3-chloro-3,3-difluoropyruvate hydrate (Oxidation of methyl 3-chloro-3,3-difluorolactate by NaClO.5H₂O)

To 4.7 mL (1 mL/mol) of acetonitrile, 0.83 g (4.7 mmol, 1.0 eq) of methyl 3-chloro-3,3-difluorolactate and 0.08 g (0.24 mmol, 0.05 eq) of tetra-n-butylammonium hydrogensulfate were added and dissolved. Furthermore, 0.94 g (5.7 mmol, 1.2 eq) of NaClO.5H₂O was added under cooling with ice, followed by stirring at room temperature for one hour. As the reaction-terminated liquid was analyzed by ¹⁹F-NMR, conversion became 100%. To the reaction-terminated liquid, 1.2 g (0.95 mmol, 0.20 eq) of 10% sodium sulfite aqueous solution was added, followed by stirring, thereby quenching the remaining oxidation agent. As the reaction liquid was quantified with internal standard method (internal standard substance: α,α,α-trifluorotoluene) by ¹⁹F-NMR, methyl 3-chloro-3,3-difluoropyruvate hydrate was contained by 4.5 mmol (quantitative yield: 95%).

¹H-NMR and ¹⁹F-NMR of methyl 3-chloro-3,3-difluoropyruvate hydrate are shown in the following.

¹H-NMR (standard substance: tetramethylsilane, solvent: deuterated chloroform), δ ppm; 3.96 (s, 3H), and assignment of proton of gem-diol group was not possible.

¹⁹F-NMR (standard substance: hexafluorobenzene; solvent: deuterated chloroform), δ ppm; 93.8 (s, 3F).

### [Example 10] Production of methyl 3,3,4,4,4-pentafluoro-2,2-dihydroxybutyrate (Oxidation of methyl 3,3,4,4,4-pentafluoro-2-hydroxybutyrate by NaClO.5H₂O)

To 1.9 mL (1 L/mol) of acetonitrile, 0.39 g (1.9 mmol, 1.0 eq) of methyl 3,3,4,4,4-pentafluoro-2-hydroxybutyrate and 0.032 g (0.091 mmol, 0.05 eq) of tetra-n-butylammonium hydrogensulfate were added and dissolved. Furthermore, 0.68 g (4.2 mmol, 2.2 eq) of NaClO.5H₂O was added under cooling with ice, followed by stirring at room temperature for 30 minutes. As the reaction-terminated liquid was analyzed by ¹⁹F-NMR, conversion became 100%. To the reaction-terminated liquid, 0.48 g (0.38 mmol, 0.20 eq) of 10% sodium sulfite aqueous solution was added, followed by stirring, thereby quenching the remaining oxidation agent. As the reaction liquid was quantified with internal standard method (internal standard substance: α,α,α-trifluorotoluene) by ¹⁹F-NMR, methyl 3,3,4,4,4-pentafluoro-2,2-dihydroxybutyrate was contained by 1.3 mmol (quantitative yield: 71%).

¹H-NMR and ¹⁹F-NMR of methyl 3,3,4,4,4-pentafluoro-2,2-dihydroxybutyrate are shown in the following.

¹H-NMR (standard substance: tetramethylsilane, solvent: deuterated chloroform), δ ppm; 3.87 (s, 3H), and assignment of proton of gem-dial group was not possible.

¹⁹F-NMR (standard substance: hexafluorobenzene; solvent: deuterated chloroform), δ ppm; 36.8 (s, 2F), 82.6 (s, 3F).

### [Example 11] Production of ethyl 3,3,3-trifluoropyruvate (Dehydration of ethyl 3,3,3-trifluoropyruvate Hydrate by Concentrated Sulfuric Acid)

5.2 g (53 mmol, 2.0 eq) of concentrated sulfuric acid was heated to 97° C. Under reduced pressure (13.5-3.3 kPa), while adding 5.0 g (27 mmol, 1.0 eq) of ethyl 3,3,3-trifluoropyruvate hydrate in a dropwise manner, a distillate was taken out, thereby obtaining 1.7 g of ethyl 3,3,3-trifluoropyruvate. ¹⁹F-NMR purity was 100% (10 mmol), and yield was 37%.

¹H-NMR and ¹⁹F-NMR of ethyl 3,3,3-trifluoropyruvate were identical with those in Japanese Patent Application Publication Showa 63-035538.

### [Example 12] Production of ethyl 3,3,3-trifluoropyruvate (Ethanol Elimination from ethyl 3,3,3-trifluoropyruvate ethylhemiketal by Concentrated Sulfuric Acid)

5.7 g (58 mmol, 4.1 eq) of concentrated sulfuric acid was heated to 97° C. Under reduced pressure (6.6-2.2 kPa), while adding 3.1 g (14 mmol, 1.0 eq) of ethyl 3,3,3-trifluoropyruvate ethylhemiketal in a dropwise manner, a distillate was taken out, thereby obtaining 1.6 g of ethyl 3,3,3-trifluoropyruvate. ¹⁹F-NMR purity was 100% (9.4 mmol), and yield was 67%.

¹H-NMR and ¹⁹F-NMR of ethyl 3,3,3-trifluoropyruvate were identical with those of Example 11.

### [Example 13] Production of methyl 3,3-difluoropyruvate (Dehydration of methyl 3,3-difluoropyruvate hydrate by trifluoroacetic Anhydride)

To 2.0 mL (0.31 mL/mmol) of cyclopentyl methyl ether, 1.0 g (6.4 mmol, 1.0 eq) of methyl 3,3-difluoropyruvate hydrate was added and dissolved therein. Furthermore, 1.1 g (14 mmol, 2.2 eq) of pyridine and 1.5 g (7.1 mmol, 1.1 eq) of trifluoroacetic anhydride were added, followed by stirring at 10° C. for one hour. As the reaction-terminated liquid was analyzed by ¹⁹F-NMR, conversion was 100%, and selectivity was 76%.

¹H-NMR and ¹⁹F-NMR of methyl 3,3-difluoropyruvate were identical with those of Example 7.

### [Example 14] Production of methyl 3,3-difluoropyruvate (Dehydration of a Mixture of methyl 3,3-difluoropyruvate Hydrate and methyl 3,3-difluoropyruvate methylhemiketal by Diphosphorus Pentoxide)

To a mixture of 0.5 g (3.3 mmol) of methyl 3,3-difluoropyruvate hydrate and 74.5 g (438 mmol) of methyl 3,3-difluoropyruvate methylhemiketal, 31.3 g (221 mmol, 0.5 eq) of diphosphorus pentoxide was slowly added at room temperature. By heat generation at this addition, the internal temperature increased to 43° C. Then, stirring was conducted at 80° C. for six hours. Then, a simple distillation (81° C./10 kPa) was conducted, thereby obtaining 54.7 g of methyl 3,3-difluoropyruvate. ¹⁹F-NMR purity was 100% (396 mmol), and yield was 90%.

¹H-NMR and ¹⁹F-NMR of methyl 3,3-difluoropyruvate were identical with those of Example 7.

### [Comparative Example 1] Oxidation of methyl 3,3-difluorolactate (Adopting the Reaction Condition of Example 6 of Patent Publication 1)

To 18 mL (2.5 mL/mmol) of methylene chloride, 1.0 g (7.1 mmol, 1.0 eq) of methyl 3,3-difluorolactate and 0.11 g (0.34 mmol, 0.048 eq) of tetra-n-butylammonium bromide were added and dissolved therein. Furthermore, 8.8 g (14 mmol, 2.0 eq) of 12 mass % sodium hypochlorite aqueous solution was added, followed by vigorous stirring at 28° C. for four hours (the reaction was a two-phase system). The reaction-terminated liquid was separated into two layers. The aqueous layer was extracted with methylene chloride, followed by a combination with the organic layer obtained by the separation. As the recovered organic layer was quantified with internal standard method (internal standard substance: α,α,α-trifluorotoluene) by ¹⁹F-NMR, methyl 3,3-difluoropyruvate hydrate was contained by 1.1 mmol. The quantitative yield was 15%. Incidentally, as the recovered aqueous layer was quantified with internal standard method (internal standard substance: potassium trifluoromethanesulfonate) by ¹⁹F-NMR, 3,3-difluoropyruvic acid hydrate and difluoroacetic acid were respectively contained by 1.6 mmol and 0.6 mmol.

¹H-NMR and ¹⁹F-NMR of methyl 3,3-difluoropyruvate hydrate were identical with those of Example 3. Furthermore, ¹⁹F-NMR of 3,3-difluoropyruvic acid hydrate is shown in the following.

¹⁹F-NMR (standard substance: trichlorofluoromethane, solvent: heavy water), δ ppm; −134.9 (d, 2F).

### [Comparative Example 2] Oxidation of 1,1,1-trifluoro-2-propanol (Adopting a Preferable Reaction Condition of the Present Invention)

To 4.4 mL (1.0 mL/mmol) of acetonitrile, 0.50 g (4.4 mmol, 1.0 eq) of 1,1,1-trifluoro-2-propanol and 0.074 g (0.22 mmol, 0.050 eq) of tetra-n-butylammonium hydrogensulfate were added and dissolved therein. Furthermore, 0.86 g (5.2 mmol, 1.2 eq) of NaClO.5H₂O was added, followed by stirring all night at room temperature. As the reaction-terminated liquid was analyzed by ¹⁹F-NMR, 1,1,1-trifluoroacetone or its hydrate was not observed at all.

### [Comparative Example 3] Oxidation of 1,1-difluoro-2-propanol (Adopting a Preferable Reaction Condition of the Present Invention)

To 5.2 mL (1.0 mL/mmol) of acetonitrile, 0.50 g (5.2 mmol, 1.0 eq) of 1,1-difluoro-2-propanol was added and dissolved therein. Furthermore, 1.0 g (6.1 mmol, 1.2 eq) of NaClO.5H₂O was added, followed by stirring all night at room temperature. As the reaction-terminated liquid was analyzed by ¹⁹F-NMR, 1,1-difluoroacetone or its hydrate was not observed at all.

### [Comparative Example 4] Oxidation of 3,3,3-trifluorolactate (Production of Trifluoroacetic Acid as a by-Product by Degradation)

To 0.85 g (5.9 mmol, 1.0 eq) of 3,3,3-trifluorolactic acid, 5.9 g (9.5 mmol, 1.6 eq) of 12 mass % sodium hypochlorite aqueous solution was added, followed by a vigorous stirring at room temperature for two hours. As the reaction-terminated liquid was analyzed by ¹⁹F-NMR, conversion was 45%, and trifluoroacetic acid was produced by 27% as a by-product. 3,3,3-trifluoropyruvic acid hydrate, which is considered to be the proper product by oxidation, was not observed at all.

### [Reference Example 1] Preparation of methyl 3,3-difluorolactate and ethyl 3,3-difluorolactate (Preparation of 3,3-difluorolactamide with Reference to Patent Publication 2)

To 190 mL (1.2 mL/mmol) of water, 20 g (160 mmol, 1.0 eq) of 3,3-difluorolactamide and 78 g (800 mmol, 5.0 eq) of sulfuric acid were added, followed by stirring at 100° C. for 20 hours. The reaction-terminated liquid was extracted with 2-methyltetrahydrofuran. The recovered organic layer was concentrated under reduced pressure, thereby obtaining 16 g (130 mmol) of 3,3-difluorolactic acid. Yield was 81%.

To 3.8 g (120 mmol, 1.5 eq) of methanol, 10 g (79 mmol, 1.0 eq) of 3,3-difluorolactic acid, 13 g (120 mmol, 1.5 eq) of trimethyl orthoformate, and 1.2 g (12 mmol, 0.15 eq) of sulfuric acid were added, followed by stirring all night at room temperature. By a simple distillation (up to 44° C./0.6 kPa) of the reaction-terminated liquid, 9.7 g (69 mmol) of methyl 3,3-difluorolactate was obtained. Yield was 87%.

¹H-NMR and ¹⁹F-NMR of methyl 3,3-difluorolactate are shown in the following.

¹H-NMR (standard substance: tetramethylsilane; solvent: deuterated chloroform), δ ppm; 3.87 (s, 3H), 4.40 (ddd, 1H), 5.96 (dt, 1H), and assignment of proton of hydroxyl group was not possible.

¹⁹F-NMR (standard substance: hexafluorobenzene; solvent: deuterated chloroform), δ ppm; 31.3 (ddd, 1F), 32.7 (ddd, 1F).

Similarly, it was possible to prepare ethyl 3,3-difluorolactate by a ethyl esterification.

¹H-NMR and ¹⁹F-NMR of ethyl 3,3-difluorolactate are shown in the following.

¹H-NMR (standard substance: tetramethylsilane; solvent: deuterated chloroform), δ ppm; 1.28 (t, 3H), 4.28 (dq, 2H), 4.35 (ddd, 1H), 5.92 (dt, 1H), and assignment of proton of hydroxyl group was not possible.

¹⁹F-NMR (standard substance: hexafluorobenzene; solvent: deuterated chloroform), δ ppm; 31.3 (ddd, 1F), 32.8 (ddd, 1F).

### [Reference Example 2] Preparation of methyl 3-chloro-3,3-difluorolactate

3-chloro-3,3-clifluorolactamide was prepared from 4.5 g (28 mmol) of 2-chloro-2,2-difluoroacetaldehyde ethyl hemiacetal by a procedure similar to Reference Example 1 with reference to Patent Publication 2. To this, 32 mL (1.1 mL/mmol) of water and 13.6 g (135 mmol, 4.8 eq) of sulfuric acid were added, followed by stirring under reflux for 80 hours. The reaction-terminated liquid was extracted with 2-methyltetrahydrofuran. The recovered organic layer was dehydrated with sodium sulfate. After filtration, the filtrate was concentrated under reduced pressure. To the obtained concentration residue, 10.3 g (321 mmol) of methanol, 4.9 g (46.1 mmol) of trimethyl orthoformate, and 0.2 g (2 mmol) of sulfuric acid were added, followed by stirring at room temperature for 22 hours. By a simple distillation (up to 48° C./2.8 kPa) of the reaction-terminated liquid, 1.7 g (9.7 mmol) of methyl 3-chloro-3,3-difluorolactate. Yield was 35%.

¹H-NMR and ¹⁹F-NMR of methyl 3,3-difluorolactate are shown in the following.

¹H-NMR (standard substance: tetramethylsilane; solvent: deuterated chloroform), δ ppm; 3.93 (s, 3H), 4.56 (dd, 1H), and assignment of proton of hydroxyl group was not possible.

¹⁹F-NMR (standard substance: hexafluorobenzene; solvent: deuterated chloroform), δ ppm; 99.5 (dd, 1F), 101. (dd, 1F).

### [Reference Example 3] Preparation of methyl 3,3,4,4,4-pentafluoro-2-hydroxybutyrate

3,3,4,4,4-pentafluoro-2-hydroxybutyric acid was synthesized from 9.0 g (49.8 mmol) of 2,2,3,3,3-pentafluoro-1-methoxy-1-propanol by a procedure similar to Reference Example 2. To this, 23.8 g (741 mmol) of methanol, 10.6 g (99.6 mmol) of trimethyl orthoformate, and 0.5 g (5.1 mmol) of sulfuric acid were added, followed by stirring all night at room temperature. By a simple distillation (up to 43° C./4.0 kPa) of the reaction-terminated liquid, 5.8 g (the target product content: 22.0 mmol) of methyl 3,3,4,4,4-pentafluoro-2-hydroxybutyrate was obtained. Yield was 44%.

¹H-NMR and ¹⁹F-NMR of methyl 3,3,4,4,4-pentafluoro-2-hydroxybutyrate are shown in the following.

¹H-NMR (standard substance: tetramethylsilane; solvent: deuterated chloroform), δ ppm; 3.93 (s, 3H), 4.56 (dd, 1H), and assignment of proton of hydroxyl group was not possible.

¹⁹F-NMR (standard substance: hexafluorobenzene; solvent: deuterated chloroform), δ ppm; 80.1 (s, 3F), 41.2 (ddd, 1F), 34.5 (ddd, 1F).

### [Reference Example 4] Study of Reactivity of ethyl 3,3,3-trifluoropyruvate methylhemiketal

To 83 mL (1.4 mL/mmol) of toluene, 12 g (59 mmol, 1.0 eq) of ethyl 3,3,3-trifluoropyruvate methylhemiketal and 3.5 g (58 mmol, 0.98 eq) of ethylenediamine were added under cooling with ice, followed by stirring at room temperature for 15 hours (crystal precipitation). As the reaction-terminated liquid was analyzed by ¹⁹F-NMR, conversion was 100%. The reaction-terminated liquid was concentrated under reduced pressure, thereby distilling a part of toluene off. The precipitated crystals were filtered, followed by washing with toluene and drying, thereby obtaining 11 g (60 mmol) of trifluoro hemiaminal amide ring closure compound represented by the following formula. Yield was quantitative.

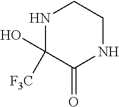 [SMILES: O=C1NCCNC1(O)C(F)(F)F]

¹H-NMR and ¹⁹F-NMR of trifluoro hemiaminal amide ring closure compound are shown in the following.

¹H-NMR (standard substance: tetramethylsilane; solvent: deuterated dimethylsulfoxide), δ ppm; 2.81 (m, 1H), 3.04 (m, 2H), 3.19 (m, 1H), 3.36 (br, 1H), 7.00 (br, 1H), 8.12 (s, 1H).

¹⁹F-NMR (standard substance: hexafluorobenzene; solvent: deuterated dimethylsulfoxide), δ ppm; 82.8 (s, 3F).

### [Reference Example 5] Study of Reactivity of methyl 3,3-difluoropyruvate methylhemiketal

To 83 mL (1.4 mL/mmol) of toluene, 10 g (59 mmol, 1.0 eq) of methyl 3,3-difluoropyruvate methylhemiketal and 3.5 g (58 mmol, 0.98 eq) of ethylenediamine were added under cooling with ice, followed by stirring at room temperature for 15 hours (crystal precipitation). As the reaction-terminated liquid was analyzed by ¹⁹F-NMR, conversion was 100%. The reaction-terminated liquid was concentrated under reduced pressure, thereby distilling a part of toluene off. The precipitated crystals were filtered, followed by washing with toluene and drying, thereby obtaining 9.8 g (59 mmol) of difluoro hemiaminal amide ring closure compound represented by the following formula. Yield was quantitative.

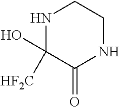

¹H-NMR and ¹⁹F-NMR of difluoro hemiaminal amide ring closure compound are shown in the following.

¹H-NMR (standard substance: tetramethylsilane; solvent: deuterated dimethylsulfoxide), δ ppm; 2.81 (m, 1H), 3.11 (m, 4H), 5.94 (t, 1H), 6.50 (s, 111), 7.96 (s, 1H).

¹⁹F-NMR (standard substance: hexafluorobenzene; solvent: deuterated dimethylsulfoxide), δ ppm; 17.7 (dd, 1F), 36.1 (dd, 1F).

### [Reference Example 6] Study of Reactivity of methyl 3,3-difluoropyruvate methylhemiketal

To 14 mL (1.5 mL/mmol) of toluene, 1.6 g (9.4 mmol, 1.0 eq) of methyl 3,3-difluoropyruvate methylherniketal and 0.58 g (9.7 mmol, 1.0 eq) of ethylenediamine were added under cooling with ice, followed by stirring at room temperature for 15 hours (crystal precipitation). Furthermore, 0.17 g (0.89 mmol, 0.095 eq) of paratoluenesulfonic acid monohydrate was added, followed by azeotropic dehydration at 130° C. for three hours using a Dean-Stark. The reaction-terminated liquid was uniformly dissolved by acetonitrile. As it was quantified with internal standard method (internal standard substance: α,α,α-trifluorotoluene) by ¹⁹F-NMR, difluoro imino amide ring closure compound represented by the following formula was contained by 0.99 g (6.7 mmol). Quantitative yield was 71%.

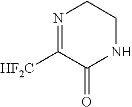

¹⁹F-NMR of difluoro imino amide ring closure compound is shown in the following.

¹⁹F-NMR (standard substance: hexafluorobenzene; solvent: deuterated chloroform), δ ppm; 38.0 (d, 2F).

### INDUSTRIAL USABILITY

Fluorine-containing α-ketocarboxylic esters, which are the targets of the present invention, are usable as intermediates of medicines and agricultural chemicals.

1.-17. (canceled)

18. A process for producing a fluorine-containing α-ketocarboxylic ester hydrate represented by the general formula [2] by reacting a fluorine-containing α-hydroxycarboxylic ester represented by the general formula [1] with sodium hypochlorite or calcium hypochlorite of 21 mass % or greater in mass percentage of composition, 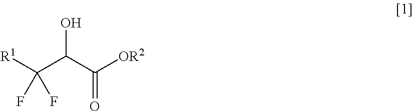
wherein R¹ represents a hydrogen atom, halogen atom or haloalkyl group, and R² represents an alkyl group or substituted alkyl group, 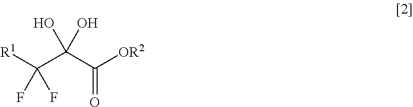
wherein R¹ represents a hydrogen atom, halogen atom or haloalkyl group, and R² represents an alkyl group or substituted alkyl group.

19. The process according to claim 18, wherein the reaction is conducted with sodium hypochlorite or calcium hypochlorite of 31 mass % or greater in mass percentage of composition.

20. The process according to claim 18, wherein the reaction is conducted with NaClO.5H₂O or Ca(ClO)₂.nH₂O wherein n represents an integer of 0 to 3.

21. The process according to claim 18, wherein R¹ of the fluorine-containing α-hydroxycarboxylic ester represented by the general formula [1] is a hydrogen atom.

22. The process according to claim 18, wherein the reaction is conducted in the presence of a phase-transfer catalyst.

23. The process according to claim 18, wherein the reaction is conducted without using a reaction solvent.

24. A process for producing a fluorine-containing α-ketocarboxylic ester represented by the general formula [3] by producing a fluorine-containing α-ketocarboxylic ester hydrate represented by the general formula [2] by the process according to claim 18 and then reacting the ester hydrate with a dehydrating agent, 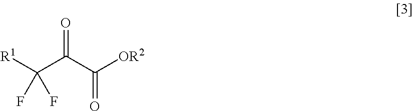
wherein R¹ represents a hydrogen atom, halogen atom or haloalkyl group, and R² represents an alkyl group or substituted alkyl group.

25. The process according to claim 24, wherein the dehydrating agent is diphosphorus pentoxide or concentrated sulfuric acid.

26. A process for producing a fluorine-containing α-ketocarboxylic ester hemiketal represented by the general formula [5] by producing a fluorine-containing α-ketocarboxylic ester hydrate represented by the general formula [2] by the process according to claim 18 and then reacting the ester hydrate with a lower alcohol represented by the general formula [4],
R³OH [4]
wherein R³ represents a C₁₋₄ alkyl group, 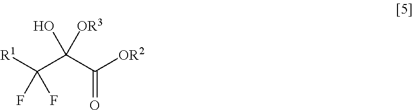
wherein R¹ represents a hydrogen atom, halogen atom or haloalkyl group, R² represents an alkyl group or substituted alkyl group, and R³ represents a C₁₋₄ alkyl group.

27. The process according to claim 26, wherein R³ of the lower alcohol represented by the general formula [4] is a methyl group or ethyl group.

28. The process according to claim 26, wherein R² of the fluorine-containing α-ketocarboxylic ester hydrate represented by the general formula [2] and R³ of the lower alcohol represented by the general formula [4] are identical alkyl groups.

29. A process for producing a fluorine-containing α-ketocarboxylic ester hemiketal represented by the general formula [5] by producing a fluorine-containing α-ketocarboxylic ester hydrate represented by the general formula [2] by the process according to claim 18 and then reacting the ester hydrate with a trialkyl orthocarboxylate represented by the general formula [6],
R⁴C(OR³)₃ [6]
wherein R³ represents a C₁₋₄ alkyl group, and R⁴ represents a hydrogen atom, methyl group or ethyl group, 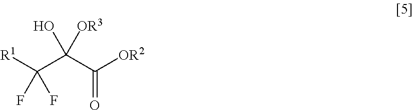
wherein R¹ represents a hydrogen atom, halogen atom or haloalkyl group, R² represents an alkyl group or substituted alkyl group, and R³ represents a C₁₋₄ alkyl group.

30. The process according to claim 29, wherein R³ of the trialkyl orthocarboxylate represented by the general formula [6] is a methyl group or ethyl group.

31. The process according to claim 29, wherein R² of the fluorine-containing α-ketocarboxylic ester hydrate represented by the general formula [2] and R³ of the trialkyl orthocarboxylate represented by the general formula [6] are identical alkyl groups.

32. The process according to claim 26, wherein the reaction is conducted in the presence of an acid catalyst.

33. A process for producing a fluorine-containing α-ketocarboxylic ester represented by the general formula [3] by producing a fluorine-containing α-ketocarboxylic ester hemiketal represented by the general formula [5] by the process according to claim 26 and then reacting the ester hemiketal with a dealcoholization agent, 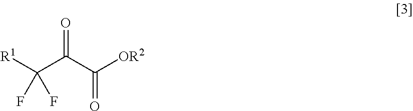
wherein R¹ represents a hydrogen atom, halogen atom or haloalkyl group, and R² represents an alkyl group or substituted alkyl group.

34. The process according to claim 33, wherein the dealcoholization agent is diphosphorus pentoxide or concentrated sulfuric acid.

35. The process according to claim 29, wherein the reaction is conducted in the presence of an acid catalyst.

36. A process for producing a fluorine-containing α-ketocarboxylic ester represented by the general formula [3] by producing a fluorine-containing α-ketocarboxylic ester hemiketal represented by the general formula [5] by the process according to claim 29 and then reacting the ester hemiketal with a dealcoholization agent, 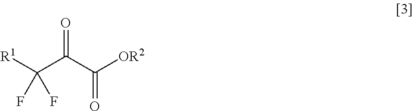
wherein R¹ represents a hydrogen atom, halogen atom or haloalkyl group, and R² represents an alkyl group or substituted alkyl group.

37. The process according to claim 36, wherein the dealcoholization agent is diphosphorus pentoxide or concentrated sulfuric acid.
